(19) United States
(12) Patent Application Publication
(10) Pub. No.: US 2023/0030812 A1
(43) Pub. Date: Feb. 2, 2023

(54) Compositions Containing Neutralized Amino Acid Esters and Glycerides

(71) Applicant: Stepan Company, Northbrook, IL (US)

(72) Inventors: Dennis S. Murphy, Libertyville, IL (US); Renata A. Butikas, Chicago, IL (US); James A. Faunce, North Aurora, IL (US); Patrick Shane Wolfe, Palatine, IL (US)

(21) Appl. No.: 17/953,520

(22) Filed: Sep. 27, 2022

Related U.S. Application Data
(63) Continuation of application No. PCT/US2021/024225, filed on Mar. 25, 2021.
(60) Provisional application No. 63/000,988, filed on Mar. 27, 2020.

(51) Int. Cl. A61K 8/44; A61K 8/37; A61Q 5/12

(52) U.S. Cl. CPC A61K 8/44; A61K 8/375; A61Q 5/12; A61K 2800/48; A61K 2800/26

(57) ABSTRACT

Compositions comprising a synergistic mixture of at least one neutralized amino acid ester and a glyceride component as a composition active are disclosed. The mixture comprises from 35% to 85% by weight neutralized amino acid ester, and from 15% to 65% by weight of a glyceride component comprising selected monoglycerides, diglycerides, or a combination thereof. The compositions are useful for hair care, as well as in other applications, such as cleaning compositions, fabric softening compositions, and skin care compositions. When formulated into a hair care formulation, the compositions provide better wet combing than either the neutralized amino acid ester or the glycerides alone. Also disclosed are end use compositions comprising the mixture of neutralized amino acid ester and selected glycerides.

### CROSS REFERENCE TO RELATED APPLICATIONS

This application is a continuation of PCT Application No. PCT/US2021/024225, filed Mar. 25, 2021, which claims priority to U.S. Provisional Application No. 63/000,988, filed Mar. 27, 2020. The entire specifications of the applications referred to above are hereby incorporated by reference.

### FIELD OF THE INVENTION

The present technology relates to compositions comprising neutralized amino acid esters and glycerides as composition actives that are useful for providing conditioning, softening, and/or cleaning properties. The compositions can be used for hair care, as well as other applications, such as cleaning compositions, fabric softening compositions, and skin care compositions. In particular, the present technology relates to compositions that comprise a synergistic mixture of at least one neutralized amino acid ester and selected glycerides that provides better wet and dry combing than either of the neutralized amino acid ester or the glycerides alone. The present technology also relates to end use compositions comprising the mixture of neutralized amino acid ester and glycerides.

### BACKGROUND OF THE INVENTION

There has been a trend in the personal care industry to formulate compositions with ingredients that are based on renewable resources derived from plants or animals, rather than fossil fuels. Such ingredients are considered “green” or “natural”, since they are derived from renewable and/or sustainable sources. As a result, they are more environmentally friendly than ingredients derived from fossil fuels, particularly if they are also manufactured without the need for petroleum-derived solvents.

An example of a natural ingredient derived from renewable sources is a neutralized amino acid ester that is obtained from the reaction product of a neutral amino acid having a non-polar side chain reacted with a long chain fatty alcohol. U.S. Pat. No. 8,105,569 describes such neutralized amino acid esters. The neutralized amino acid esters are cationic, and therefore could potentially replace traditional cationic hair conditioning agents, such as behentrimonium chloride (BTAC) and cetrimonium chloride (CETAC), which traditionally have unfavorable environmental profiles.

One drawback of the neutralized amino acid ester is that it is a more expensive cationic ingredient than other cationic components typically used in hair care compositions, such as quaternary ammonium compounds and amidoamines. In addition, a greater amount of the neutralized amino acid ester, compared to the traditional cationic active agents, is often required to achieve acceptable performance. Using more of an ingredient that is already more costly results in a more expensive product to manufacture. It would therefore be desirable to have composition actives that are derived from renewable sources, but that can also deliver acceptable performance at lower cost, and at use levels comparable to the use levels used for traditional cationic active agents.

### SUMMARY OF THE INVENTION

The present technology is directed to compositions comprising a synergistic mixture of at least one neutralized amino acid ester and a glyceride component, wherein the glyceride component comprises monoglycerides, diglycerides, or mixtures thereof, and optionally, from 0 to 50% by weight triglycerides, based on the total weight of the glycerides. Surprisingly, it has been found that combining particular glycerides with the neutralized amino acid ester can improve the wet combing properties of the neutralized amino acid ester.

In one aspect, the present technology is directed to a composition comprising:

30%-100% by weight of the composition of a mixture comprising:
(a) a neutralized amino acid ester selected from the group consisting of Brassica L-isoleucine esylate, leucine isostearyl ester esylate, and combinations thereof, in an amount of 35%-85% by weight of the mixture; and
(b) a glyceride component in an amount of 15%-65% by weight of the mixture, wherein the glyceride component comprises monoglycerides, diglycerides, or a combination thereof, and optionally from 0 to 50% by weight triglycerides, based on the weight of the glyceride component, provided that, if the only glyceride in the glyceride component is a monoglyceride, that monoglyceride has a fatty acid carboxylate group of greater than 10 carbon atoms; and
optionally, 0-70% by weight of the composition of one of more solvents.

In another aspect, the present technology is directed to a composition comprising:
(a) 0.01% to about 50% by weight of a composition active, wherein the composition active comprises a mixture of (i) a neutralized amino acid ester selected from the group consisting of Brassica L-isoleucine esylate, leucine isostearyl ester esylate, and combinations thereof, in an amount of 35%-85% by weight of the mixture, and (ii) a glyceride component in an amount of 15% to 65% by weight of the mixture, wherein the glyceride component comprises monoglycerides, diglycerides, or a combination thereof, and optionally, 0 to 50% by weight triglycerides, based on the weight of the glyceride component, provided that, if the only glyceride in the glyceride component is a monoglyceride, that monoglyceride has a fatty acid carboxylate group of greater than 10 carbon atoms;
(b) optionally, one or more additional components; and
(c) diluent to balance the formulation to 100%. In one embodiment, the formulation is a hair conditioning composition.

### DETAILED DESCRIPTION OF THE PREFERRED EMBODIMENTS

The compositions of the present technology comprise a mixture of particular neutralized amino acid esters and particular glycerides that together provide a synergistic mixture of actives that is derived from renewable sources, is cost effective, and provides conditioning, softening, or cleaning performance that is better than either the neutralized amino acid ester or glycerides alone at comparable use levels.

The neutralized amino acid ester of the present technology is obtained by esterification of (i) an amino acid having a non-polar side chain wherein the amine group of the amino acid has been neutralized with an acid; with (ii) a long chain fatty alcohol. The amino acid ester of the present technology may be represented by the structure of formula (I):

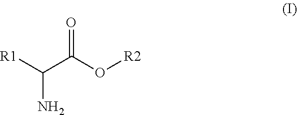

wherein R¹ represents an alkyl group, that may be branched or linear, from one to ten carbon atoms, alternatively from two to six carbon atoms. R² represents a carbon chain that may be linear or branched, saturated or unsaturated. The carbon chain may have from eight to fifty carbon atoms, alternatively eight to thirty-two carbon atoms, alternatively eight to twenty-four carbon atoms. Amino acids for the formation of the ester include any that are neutral. Particular amino acids include L-alanine, L-valine, L-leucine and L-isoleucine. In some embodiments, the amino acid, is L-isoleucine or L-leucine.

To obtain the neutralized amino acid ester, the amine group of the amino acid is neutralized with an acid, and is reacted with a long chain fatty alcohol. Suitable fatty alcohols may be linear or branched and may additionally be saturated and/or unsaturated. It may be preferred that the fatty alcohol contains about 10 to about 50, alternatively about 24 to about 32 carbon atoms. In some embodiments, linear and/or branched fatty alcohols containing from about 12 to about 22 carbon atoms may be preferred. Examples of suitable fatty alcohols include lauryl alcohol, myristyl alcohol, palmityl alcohol, stearyl alcohol, oleyl alcohol, isostearyl alcohol, arachidyl alcohol, behenyl alcohol, or mixtures or combinations thereof. Preferably, the fatty alcohols are derived from non-petrochemical sources.

The amine group of the amino acid ester may be fully or partially neutralized by an acid, to facilitate its cationic behavior. Any acid may be used, including organic and inorganic acids. Suitable acids include, without limitation, mineral acids, amino acids, hydrochloric acid, phosphoric acid, sulfuric acid, boric acid, and nitric acid. Suitable organic acids may be citric acid, ethane sulfonic acid, acetic acid, formic acid, or oxalic acid. Suitable amino acids may include glutamic acid and aspartic acid. In some embodiments, the acid is ethane sulfonic acid. Preferred neutralized amino acid esters are Brassicyl L-isoleucine esylate, or leucine isostearyl ester esylate. Brassicyl L-isoleucine esylate may be derived from the esterification of Brassica alcohol with L-isoleucine esylate. L-isoleucine esylate may be prepared by reacting the amine group on isoleucine with ethanesulfonic acid. Brassica alcohol is a fatty alcohol that is derived from the splitting of high erucic acid rapeseed (HEAR) oil obtained from the Brassica genus of plants, followed by hydrogenation. Brassica alcohol consists predominantly of stearyl, arachidyl, and behenyl alcohols with minor quantities of lower and higher alkyl chain length alcohols. The neutralized amino acid ester of the present technology may be synthesized by methods commonly known in the art.

In addition to the neutralized amino acid esters, compositions of the present technology comprise a glyceride component. The glyceride component may comprise monoglycerides, diglycerides, or mixtures thereof. Optionally, triglycerides may also be included in the glyceride component. An amount of triglycerides in the glyceride component can range from 0 to about 50% by weight, alternatively 0 to about 40% by weight, alternatively 0 to about 30% by weight, alternatively 1% to about 50% by weight, alternatively about 1% to about 40%, about 1% to about 30%, about 1% to about 20%, or about 1% to about 10% by weight, based on the total weight of the glyceride component. The monoglycerides, diglycerides, or triglycerides, or combinations thereof, comprise saturated, unsaturated, or a mixture of unsaturated and saturated fatty acid carboxylate groups containing about 8 to about 32 carbon atoms. However, if the glyceride component comprises only or primarily monoglycerides, the fatty acid carboxylate groups of the monoglycerides should have greater than 10 carbon atoms. In some embodiments, the fatty acid groups comprise at least 50% by weight, alternatively at least 60% by weight, unsaturated fatty acid groups having at least one carbon-carbon double bond. In some embodiments, the fatty acid groups are derived from oleic acid. In some embodiments, the glyceride component is a mixture of monoglycerides and diglycerides. The ratio of monoglyceride to diglyceride in the mixture can be about 1:3 to 3:1, although in some embodiments, a ratio of about 1:1 monoglyceride to diglyceride is preferred. The mixture of neutralized amino acid ester active and glyceride component comprises about 35% to 85%, alternatively about 40% to about 80%, alternatively about 45% to about 70%, alternatively about 50% to about 60% by weight, of neutralized amino acid ester, and about 15% to about 65%, alternatively about 20% to about 40%, alternatively about 30% to about 55%, alternatively about 40% to about 50% by weight of the glyceride component, based on the combined weight of the neutralized amino acid ester active and glyceride component. In some embodiments, the mixture of neutralized amino acid ester active and glyceride component comprises about 55% to 57% by weight of neutralized amino acid ester active, and the glyceride component comprises about 43% to 45% by weight, based on the combined weight of the neutralized amino acid ester active and glyceride component.

The mixture of neutralized amino acid ester and glycerides can be used alone as an active component, or diluted in particular solvents to form a diluted neutralized amino acid ester/glyceride composition. In some embodiments, the solvents are those suitable for personal care. Examples of solvents for diluting the neutralized amino acid ester/glyceride mixture include, but are not limited to, propylene glycol, 1,3-propandiol, glycol ethers, glycerin, sorbitan esters, lactic acid, alkyl lactyl lactates, isopropyl alcohol, ethyl alcohol, dimethyl adipate, oleyl alcohol, 1,2-isopropylidine glycerol, benzyl alcohol, dimethyl lauramide myristamide, N-butyl lactate, trimethyl citrate, dimethyl lactide, laureth-2 lactide, 1,2-butylene carbonate, conjugated linoleic acid, isosorbide dimethyl ether, propylene carbonate, C6-C18 methyl esters, citrate, C12-15 alkyl benzoate, or combinations thereof.

Although glycerides can also be used as solvents in some compositions, any glyceride in the compositions of the present technology is considered to be only a glyceride and should not be considered a solvent or counted as part of the solvent system. This definition applies regardless of where the glyceride is added in the process of making the composition. Glycerides include, but are not limited to, glyceryl esters, monoglycerides, diglycerides, triglycerides, glycerol monooleate, vegetable oil, sunflower oil, jojoba oil, borage oil, moringa oil, argan oil, or radish seed oil.

When used, the amount of solvent can range from about 1% to about 70%, alternatively about 5% to about 70%, alternatively about 10% to about 60%, alternatively about 10% to about 50%, alternatively about 10% to about 40%, alternatively about 10% to about 30% by weight, and the amount of the mixture of neutralized amino acid ester/glycerides can range from about 30% to 99%, alternatively about 30% to about 95%, alternatively about 40% to about 90%, alternatively about 50% to about 90%, alternatively about 60% to about 90%, alternatively about 70% to about 90% by weight of the composition. In some embodiments, the amount of solvent is about 1% to about 50% by weight, and the amount of the neutralized amino acid ester active and glycerides mixture is about 50% to about 99% by weight.

The diluted neutralized amino acid ester/glycerides composition can be prepared by first mixing the neutralized amino acid ester with the glyceride component to form a mixture, and then diluting the mixture in the solvent to form the diluted composition. Alternatively, the neutralized amino acid ester and glycerides can be separately combined with the solvent to form the diluted composition.

The compositions comprising the mixture of neutralized amino acid ester and glyceride component of the present technology can advantageously be formulated into hair care compositions including, but not limited to, hair conditioners and hair repair compositions. The compositions could also be formulated into other end use products such as, but not limited to, fabric softeners, fabric conditioners, hard surface cleaners, and skin care compositions. Product formulations can include the mixture of neutralized amino acid ester active and glyceride component in an amount of about 0.01% to about 50% by weight of the product formulation, alternatively about 0.05% to about 25%, alternatively about 0.1% to about 12%, alternatively about 0.01% to about 10%, alternatively about 0.1% to about 5%, alternatively about 0.5% to about 5%, alternatively about 1% to about 5%, alternatively about 1% to about 4% by weight of the product formulation.

The product formulations may contain other optional ingredients suitable for use, such as surfactants or other additives, and a diluent, such as water. Examples of surfactants include nonionic, cationic, and amphoteric surfactants, or combinations thereof. Examples of nonionic surfactants include, but are not limited to, fatty alcohol alkoxylates, polyalkylene glycols, mono- and/or dialkyl sulfosuccinates, fatty acid isethionates, fatty acid sarcosinates, fatty acid glutamates, ether carboxylic acids, alkyl oligoglucosides, and combinations thereof. Examples of cationics include, but are not limited to, BTAC, CETAC, and polyquaterniums, or combinations thereof. Examples of amphoteric surfactants include, but are not limited to, betaines, amidopropylbetaines, or combinations thereof. Other contemplated components include the long chain amido amines, such as stearamidopropyl dimethylamine (SAPDMA). Surfactant amounts in the product formulation can range from about 0.01% to about 20% by weight of the product formulation.

Examples of additives include rheological modifiers, emollients, skin conditioning agents, emulsifier/suspending agents, thickeners, fragrances, colors, opacifiers, herbal extracts, vitamins, builders, enzymes, preservatives, antibacterial agents, pH adjusters, or combinations thereof. Particular examples of such additives include, but are not limited to, silicones, siloxanes, mineral oils, natural or synthetic waxes, polyglycerol alkyl esters, glycol esters, esters of fatty acids with alcohols of low carbon number, for example isopropanol, benzoic acid esters, citric acid, succinic acid, phosphoric acid, sodium hydroxide, sodium carbonate, vitamins, such as Vitamin A, Vitamin E, or pantothenic acid, quaternized guar, celluloses or quaternized celluloses, or combinations of any of the foregoing. Total additives in the product formulation can range from about 0.01% to about 20% by weight of the product formulation.

Compositions of the present technology, comprising the mixture of neutralized amino acid ester and glyceride component, provide several benefits. Both the neutralized amino acid ester and glyceride component can be derived from natural sources, and therefore are more environmentally friendly than components derived from petroleum sources. The hair conditioning formulations comprising the compositions provide better wet hair combing properties compared to formulations comprising neutralized amino acid esters without the selected glyceride component, at comparable use levels. Since the glyceride component is a less costly ingredient than the neutralized amino acid ester, the mixture of the neutralized amino acid ester and glyceride component can provide comparable or better performance properties at a lower cost, compared to formulations comprising the neutralized amino acid ester without the glyceride component. The hair conditioning formulations also provide better wet hair combing properties compared to formulations comprising CETAC. However, unlike CETAC, the neutralized amino acid ester and glyceride component mixture is biodegradable, and provides an improved environmental profile and lower toxicity compared to CETAC.

Hair conditioning compositions comprising the mixture of neutralized amino acid ester and glyceride component of the present technology can be applied to the hair in an amount suitable for obtaining a hair conditioning effect. Suitable amounts of the mixture as a conditioning active applied to the hair can range from about 0.001% to about 5% by weight, alternatively about 0.001% to about 2%, alternatively about 0.002% to about 1.5%, alternatively about 0.025% to about 0.5%, alternatively about 0.025% to about 0.25% by weight, as measured on dry hair. The hair conditioning compositions provide a wet combing Dia-Stron Maximum Peak Load of about 80 gram mass force (gmf) or less, alternatively about 70 gmf or less, alternatively about 50 gmf or less, such as about 30 to about 50 gmf.

### EXAMPLES

The presently described technology and its advantages will be better understood by reference to the following examples. These examples are provided to describe specific embodiments of the present technology. By providing these examples, the inventors do not limit the scope and spirit of the present technology.

The following test methods are used to determine properties and performance of compositions of the present technology.

### Dia-Stron Procedure for Wet and Dry Combing

1. Rinse tress for 30 seconds.
2. Apply 0.5 mL of VO5® Volumizing Shampoo (non-conditioning shampoo).
3. Spread throughout tress.
4. Allow to air-dry.
5. Rinse tress for 30 seconds.
6. Apply 0.5 mL of Test Conditioner.
7. Spread throughout tress.
8. Affix tress to Dia-Stron MT1775 instrument and run “Wet Combing” procedure.
9. Repeat Step 8 nine more times.
10. Repeat Steps 1-9 for 2 more tresses.
11. Allow tresses to air-dry.
12. Affix tress to Dia-Stron MT1775 instrument and run “Dry Combing” procedure.
13. Repeat Step 12 nine more times for one tress.
14. Repeat Steps 12-13 for 2 more tresses.

### Example 1

Brassicyl L-Isoleucine Esylate was prepared according to the procedure of Example 1 of U.S. Pat. No. 8,105,569 to Burgo. The Example 1 procedure is hereby incorporated by reference. The reaction product, named RXN1, was measured by NMR to be 54.7% by weight Brassicyl L-Isoleucine esylate (“BISL”).

### Example 2: Preparation of Hair Conditioning Composition

Cationic actives according to the present technology, comprising the combination of at least one neutralized amino acid ester and glycerides, were formulated into hair conditioning compositions following the general procedure set forth below. Hair conditioning compositions were also formulated using comparative cationic hair conditioning components. Table 1 shows the general formula used to make the hair conditioning compositions.

TABLE 1

| Material | | % W/W in |
|---|---|---|
| Chemical Name | Function | Formulation |
| Deionized Water | Carrier | q.s. to 100.0′ |
| Natrosol ™ 250 HHR CS | Thickener | 0.7 |
| (Hydroxyethylcellulose) | | |
| Sodium Hydroxide | pH adjuster | q.s. |
| Conditioning Agent | Conditioning | Per examples which follow |
| | Active | |
| Cetyl Alcohol | Viscosity | 2.0 |
| | Modifier | |
| Potassium Chloride, 10% | Opacifier | 0.5 |
| Solution | | |
| Citric Acid | pH adjuster | q.s. |
| Kathon ™ CG | Preservative | q.s. |

### General Procedure

1. Charge water, begin mixing
2. Sprinkle in Natrosol 250 HHR CS
3. Adjust pH with 25% Sodium Hydroxide to target of pH 8-9. Mix until clear (30-40 min)
4. Heat to 70-75° C.
5. Add Conditioning component and mix until homogenous
6. Add Cetyl Alcohol and mix for 30 min.
7. Cool to 45° C. with mixing
8. In a small beaker dissolve Potassium Chloride in Water. Add to batch
9. Adjust pH 3.5-4 with 50% Citric Acid
10. Cool to Room Temp.
11. Add Kathon CG

The hair conditioning formulations used in the following examples were prepared in accordance with the Table 1 formulation and the General Procedure. Amounts in the following Tables are based on weight %. BTAC refers to behentrimonium chloride, CETAC refers to cetrimonium chloride (AMMONYX® CETAC-30 from Stepan Company, Northfield, Ill.), GMO refers to DREWMULSE® GMO, a glycerol oleate comprising mono- and diglyceryl oleates in a ratio of about 1:1, NEOBEE® M-5 COSMETIC or M-5 refers to caprylic/capric triglycerides, and STEPAN-MILD® GCC or GCC refers to caprylic/capric glycerides, primarily caprylic/capric monoglycerides, all from Stepan Company, Northfield, Ill. Each of the hair conditioning compositions was evaluated for wet combing ability using the Dia-Stron MTT175 instrument and the wet combing procedure.

### Example 3: Comparative Conditioning Actives

Comparative hair conditioning formulations were prepared according to the general formula in Table 1, using BTAC or CETAC as the conditioning active, and the amounts of conditioning active used are shown in Table 2. The formulations were evaluated for wet combing ability, and the results are provided in Table 2.

TABLE 2

| | | Amount of | | |
|---|---|---|---|---|
| | | “as is” | | |
| | % Active in | conditioning | Total | |
| Condi- | condi- | agent used in | active in | Maximum |
| tioning | tioning | formula | formula | peak load |
| Agent | agent | (%) | (%) | (gmf) |
| BTAC | 70 | 2.86 | 2.0 | 19.9 |
| CETAC | 30 | 6.67 | 2.0 | 68.7 |

The results show that commonly used conditioning actives, namely BTAC and CETAC, yield Dia-Stron maximum peak load results of about 20 gmf and 70 gmf, respectively, when used at 2% active in a typical formula.

### Example 4: Inventive, Synergistic Conditioning Agent

Hair conditioning formulations were prepared to assess whether combining glycerides with a neutralized amino acid ester can improve the wet combing properties of the hair care formulation compared to formulations containing the neutralized amino acid ester alone, or the glyceride alone, as the conditioning agent. Hair conditioning formulations were prepared using the Table 1 formulation, and using the Brassicyl L-Isoleucine esylate reaction product of Example 1 (RXN1) alone as a conditioning agent, GMO alone as a conditioning agent, and a mixture of RXN1 and GMO as the conditioning agent. The amounts of the conditioning agent used in each formulation are shown in Table 3. Each of the hair conditioning formulations was evaluated for wet combing ability using the Dia-Stron wet combing procedure. The results are shown in Table 3.

TABLE 3

| | | Amount of | | |
|---|---|---|---|---|
| | | “as is” | | |
| | % Active in | conditioning | Total | |
| Condi- | condi- | agent used in | active in | Maximum |
| tioning | tioning | formula | formula | peak load |
| Agent | agent | (%) | (%) | (gmf) |
| RXN1 | 54.7 BISL | 3.66 | 2.0 BISL | 201.3 |
| Mixture of 70% | 54.7 BISL in | 1.4 of RXN1 | 0.77 of BISL | 49.8 |
| RXN1 and 30% | RXN1 | 0.6 of GMO | 0.6 of GMO | |
| GMO (by weight) | 100 in GMO | | Total = 1.37 | |
| GMO | 100 | 2.0 | 2.0 | 62 |

This example shows that at lower overall actives (1.37% by weight), a mixture of BISL and GMO gives better hair conditioning than either component alone. In this example, there is 56.2% BISL (0.77/1.37×100=56.2%) and 43.8% GMO (0.6/1.37×100=43.8%) in the active mixture.

The results demonstrate that combining glycerides with a neutralized amino acid ester provides a synergistic mixture that imparts improved properties to a hair conditioning composition when compared to either the neutralized amino acid ester alone or the glycerides alone. It will also be appreciated that the glycerides are a less costly ingredient that the neutralized amino acid ester. Thus, the mixture of glycerides with the neutralized amino acid ester not only improves the wet combing properties of the cationic active, but also reduces the cost of the cationic active in the hair care composition. The example also shows that, in comparing the results in Table 3 with those in Table 2, the combination of neutralized amino acid ester and glycerides can provide better hair conditioning than CETAC, a common cationic conditioning active, even using a lower amount of the neutralized amino acid ester/glycerides mixture as the conditioning active.

### Example 5: Comparative Formulations

In this example, different hair conditioning formulations were prepared to assess the effect of replacing the mono- and diglyceride component (GMO) with other common glyceride conditioning agents. Hair conditioning formulations were prepared using the Table 1 formulation, and the type and amount of conditioning agent used in each formulation is shown below in Table 4. Each of the hair conditioning formulations was evaluated for wet combing ability using the Dia-Stron MTT175 instrument and the wet combing procedure. The results are shown in Table 4.

TABLE 4

| | | Amount of | | |
|---|---|---|---|---|
| | | “as is” | | |
| | % Active in | conditioning | Total | |
| Condi- | condi- | agent used in | active in | Maximum |
| tioning | tioning | formula | formula | peak load |
| Agent | agent | (%) | (%) | (gmf) |
| Mixture of 60% | 54.7 BISL in | 1.2 of RXN1 | 0.66 of BISL | 122.2 |
| RXN1 and 40% | RXN1 | 0.8 of M-5 | 0.8 of M-5 | |
| NEOBEE ® M-5 | 100 in M-5 | | Total = 1.46 | |
| Cosmetic (by | | | | |
| weight) | | | | |
| Mixture of 65% | 54.7 BISL in | 1.3 of RXN1 | 0.71 of BISL | 113.4 |
| RXN1 and 35% | RXN1 | 0.7 of | 0.7 of Olive | |
| Olive Oil (by | 100 in Olive | Olive Oil | Oil | |
| weight) | Oil | | Total = 1.41 | |
| Mixture of 65% | 54.7 BISL in | 1.3 of RXN1 | 0.71 of BISL | 97.1 |
| RXN1 and 35% | RXN1 | 0.7 of GCC | 0.7 of GCC | |
| GCC (by weight) | 100 in GCC | | Total = 1.41 | |

This example demonstrates that if the alkyl chain lengths of the glyceride are too short, for example C8-C10 monoglycerides (GCC), or C8-C10 triglycerides (Neobee® M-5 cosmetic), or if there is not enough mono and diglyceride present (as in the case of olive oil), the BISL/glyceride system does not condition well (i.e. the maximum peak load is above 95 gmf).

When comparing the results from Table 3 with those in Table 4, the results suggest, without being bound by theory, that the amount of mono- and diglyceride present in the glyceride component may have an effect on the wet combing properties. The results also suggest that carbon chain distribution and/or unsaturation in the glyceride component may have an effect on the wet combing performance of the glyceride/neutralized amino acid ester mixture.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims.

1. A composition comprising:
30%-100% by weight of the composition of a mixture comprising:
(a) a neutralized amino acid ester selected from the group consisting of Brassica L-isoleucine esylate, leucine isostearyl ester esylate, and combinations thereof, in an amount of 35%-85% by weight of the mixture; and
(b) a glyceride component in an amount of 15%-65% by weight of the mixture, wherein the glyceride component comprises monoglycerides, diglycerides, or a combination thereof, and optionally up to 50% by weight triglycerides, based on the weight of the glyceride component, provided that, if the only glyceride in the glyceride component is a monoglyceride, that monoglyceride has a fatty acid carboxylate group of greater than 10 carbon atoms; and
optionally, 0-70% by weight of the composition of one of more solvents.

2. The composition of claim 1, wherein an amine group of the amino acid is neutralized by an acid selected from the group consisting of ethane sulfonic acid, hydrochloric acid, sulfuric acid, phosphoric acid, lactic acid, citric acid, maleic acid, adipic acid, boric acid, glutamic acid, glycolic acid, acetic acid, ascorbic acid, uric acid, oxalic acid, aspartic acid, butyric acid, lauric acid, glycine, formic acid, and combinations thereof.

3. The composition of claim 1, wherein the glyceride component comprises a combination of monoglycerides and diglycerides having a carbon chain length of 8-32 carbon atoms.

4. The composition of claim 3, wherein the monoglycerides and diglycerides have a carbon chain length of 16-22 carbon atoms.

5. The composition of claim 1, wherein at least 50% of the carbon chains in the monoglycerides and diglycerides in the glyceride component have at least one double bond.

6. The composition of claim 1, wherein the monoglycerides and diglycerides are present in the glyceride component in a ratio of 3:1 to 1:3.

7. The composition of claim 1, wherein the neutralized amino acid ester comprises about 50% to about 60% by weight of the mixture, and the glyceride component comprises about 40% to about 50% by weight of the mixture.

8. The composition of claim 1, wherein the solvent is selected from the group consisting of propylene glycol, 1,3-propandiol, glycol ethers, glycerin, sorbitan esters, lactic acid, alkyl lactyl lactates, isopropyl alcohol, ethyl alcohol, dimethyl adipate, oleyl alcohol, 1,2-isopropylidine glycerol, benzyl alcohol, dimethyl lauramide myristamide, N-butyl lactate, trimethyl citrate, dimethyl lactide, laureth-2 lactide, 1,2-butylene carbonate, conjugated linoleic acid, isosorbide dimethyl ether, propylene carbonate, C6-C18 methyl esters, citrate, C12-15 alkyl benzoate, and combinations thereof.

9. A hair care composition comprising:
(a) about 0.01% to about 50% by weight of a composition active, wherein the composition active comprises a mixture of (i) a neutralized amino acid ester selected from the group consisting of Brassica L-isoleucine esylate, leucine isostearyl ester esylate, and combinations thereof, in an amount of 35%-85% by weight of the mixture, and (ii) a glyceride component in an amount of 15% to 65% by weight of the mixture, wherein the glyceride component comprises monoglycerides, diglycerides, or a combination thereof, and optionally up to 50% by weight triglycerides, based on the weight of the glyceride component, provided that, if the only glyceride in the glyceride component is a monoglyceride, that monoglyceride has a fatty acid carboxylate group of greater than 10 carbon atoms;
(b) optionally, one or more additional components; and
(c) diluent to balance the formulation to 100%.

10. The composition of claim 9, wherein an amine group of the amino acid is neutralized by an acid selected from the group consisting of ethane sulfonic acid, hydrochloric acid, sulfuric acid, phosphoric acid, lactic acid, citric acid, maleic acid, adipic acid, boric acid, glutamic acid, glycolic acid, acetic acid, ascorbic acid, uric acid, oxalic acid, aspartic acid, butyric acid, lauric acid, glycine, formic acid, and combinations thereof.

11. The composition of claim 9, wherein the glyceride component comprises a combination of monoglycerides and diglycerides having a carbon chain length of 8-32 carbon atoms.

12. The composition of claim 11, wherein the monoglycerides and diglycerides have a carbon chain length of 16-22 carbon atoms.

13. The composition of claim 9, wherein at least 50% of the carbon chains in the monoglycerides and diglycerides in the glyceride component have at least one double bond.

14. The composition of claim 9, wherein the monoglycerides and diglycerides are present in the glyceride component in a ratio of 3:1 to 1:3.

15. The composition of claim 9, wherein the neutralized amino acid ester comprises about 50% to about 60% by weight of the mixture, and the glyceride component comprises about 40% to about 50% by weight of the mixture.

16. The composition of claim 9, wherein the composition active further comprises a solvent selected from the group consisting of propylene glycol, 1,3-propandiol, glycol ethers, glycerin, sorbitan esters, lactic acid, alkyl lactyl lactates, isopropyl alcohol, ethyl alcohol, dimethyl adipate, oleyl alcohol, 1,2-isopropylidine glycerol, benzyl alcohol, dimethyl lauramide myristamide, N-butyl lactate, trimethyl citrate, dimethyl lactide, laureth-2 lactide, 1,2-butylene carbonate, conjugated linoleic acid, isosorbide dimethyl ether, propylene carbonate, C6-C18 methyl esters, citrate, C12-15 alkyl benzoate, and combinations thereof.

17. The composition of claim 16, wherein the solvent amount in the composition active is about 1% to about 70% by weight.

18. The composition of claim 9, wherein the hair care composition, when applied to a hair tress, provides a wet combing Dia-Stron maximum peak load of about 50 gram mass force (gmf) or less.

19. The composition of claim 9, wherein the diluent comprises water, a solvent, or a combination thereof.

20. The composition of claim 9, wherein the composition active is present in the composition in an amount of 0.01% to about 12% by weight of the composition.
